(19) United States
(12) Patent Application Publication
(10) Pub. No.: US 2011/0319340 A1
(43) Pub. Date: Dec. 29, 2011

(54) METHODS FOR TREATING CANCER

(75) Inventor: Darrell H. Carney, Dickinson, TX (US)

(73) Assignee: The Board of Regents, The University of Texas System

(21) Appl. No.: 13/119,926

(22) PCT Filed: Sep. 18, 2009
(86) PCT No.: PCT/US2009/057571

§ 371 Date: Sep. 6, 2011

Related U.S. Application Data
(60) Provisional application No. 61/192,686, filed on Sep. 19, 2008.

(51) Int. Cl. A61K 38/16; A61P 35/00; A61P 35/04; A61K 38/10

(52) U.S. Cl. 514/19.8; 514/19.3

(57) ABSTRACT

The present invention includes methods of treating cancer in a subject, comprising administering to the subject a therapeutically effective amount of a non-proteolytically activated thrombin receptor agonist.

### CROSS-REFERENCE TO RELATED APPLICATION

This application claims benefit under 35 U.S.C. §119(e) of U.S. Provisional Application No. 61/192,686, filed Sep. 19, 2008, which is herein incorporated by reference in its entirety.

### BACKGROUND OF THE INVENTION

Cancer is a class of diseases in which abnormal cells undergo uncontrolled cell division and are able to invade other tissues. Cancer is though to arise in some cases from a mutation in a gene which regulates cell growth and division. The mutation in the DNA is generally caused by external agents and/or though inherited genetic factors. Cancer can further progress into malignancy in which it can spread to another part of the body through a process called “metastasis.” Metastases are known to be the main cause of death from cancer.

There are more than 100 types of cancers, typically classified by organ, tissue, or type of cells in which the cancer cell originates. The most common cancer include carcinoma (cancer that begins in the skin or in tissues that line or cover internal organs); sarcoma (cancer that begins in bone, cartilage, fat, muscle, blood vessels, or other connective or supportive tissue); leukemia (cancer that starts in blood-forming tissue such as the bone marrow and causes large numbers of abnormal blood cells to be produced and enter the blood); lymphoma and myeloma (cancers that begin in the cells of the immune system); and central nervous system cancers (cancers that begin in the tissues of the brain and spinal cord).

According to World Health Organization (WHO), cancer is a worldwide leading cause of death and accounts for approximate 13% of all deaths. The mortality rate of cancer depends on the type of cancer. Lung, stomach, liver, colon and breast cancer cause the most cancer deaths each year. Among men, lung, stomach, liver, colorectal, esophageal, and prostate cancers are seen most frequently. Among women, breast, lung, stomach, colorectal and cervical cancer are most frequent.

Despite intensive scientific efforts to elucidate the molecular mechanisms, development of effective anti-cancer therapies which can reduce, limit or inhibit metastasis of cancer cells has been largely elusive. There is a great need for an agent which can provide an effective treatment for cancer.

### SUMMARY OF THE INVENTION

TP508, a polypeptide which stimulates or activates a non-proteolytically activated thrombin receptor (hereinafter “NPAR”), can be used to treat cancer. TP508 can inhibit cancer metastasis and/or proliferation. The present invention includes methods of treating cancer in a subject (e.g., a human patient) having cancer, comprising administering to the subject a therapeutically effective amount of an NPAR agonist. In some embodiments, there is provided a method of reducing or preventing metastasis of cancer in a subject by administering to the subject a therapeutically effective amount of an agonist of NPAR. In some embodiments, the cancer is colon cancer.

In the methods of the invention, the NPAR agonist is a thrombin peptide derivative disclosed herein. More specifically, one thrombin peptide derivative comprises the polypeptide Arg-Gly-Asp-Ala-Cys-X₁-Gly-Asp-Ser-Gly-Gly-Pro-X₂-Val (SEQ ID NO:1), or a C-terminal truncated fragment thereof comprising at least six amino acid residues. In another specific embodiment, the thrombin peptide derivative comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), an N-terminal truncated fragment of the thrombin peptide derivative having at least fourteen amino acid residues, or a C-terminal truncated fragment of the thrombin peptide derivative comprising at least eighteen amino acid residues. X₁ is Glu or Gln and X₂ is Phe, Met, Leu, His or Val. In another specific embodiment, the thrombin peptide derivative is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3).

In another embodiment, the NPAR agonist is a modified thrombin peptide derivative disclosed herein. In a specific embodiment, the modified thrombin peptide derivative comprises the polypeptide Arg-Gly-Asp-Ala-Xaa-X₁-Gly-Asp-Ser-Gly-Gly-Pro-X₂-Val (SEQ ID NO:4), or a C-terminal truncated fragment thereof having at least six amino acid residues. In another specific embodiment, the modified thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5), or a fragment thereof comprising amino acid residues 10-18 of SEQ ID NO:5.

The pharmaceutical compositions comprising thrombin peptide derivatives or modified thrombin peptide derivatives of the present invention can also include a dimerization inhibitor. A dimerization inhibitor is a compound that inhibits or reduces dimerization of a thrombin peptide derivative or a modified thrombin peptide derivative. Dimerization inhibitors include chelating agents and/or thiol-containing compounds.

In another embodiment, the NPAR agonist is a dimer of two thrombin peptide derivatives disclosed herein. More specifically, one such dimer comprises the amino acid sequence Arg-Gly-Asp-Ala-Cys-X₁-Gly-Asp-Ser-Gly-Gly-Pro-X₂-Val (SEQ ID NO:1) or a C-terminal truncated fragment thereof having at least six amino acid residues. In another specific embodiment, the dimer comprises a polypeptide having the amino acid sequence Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), or a fragment thereof comprising amino acid residues 10-18 of SEQ ID NO:2. In another embodiment of the invention, the dimer comprises the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3). In another specific embodiment, the dimer is represented by the structural formula (IV).

The thrombin referred to above can be a mammalian thrombin, and in particular, a human thrombin. The portion of thrombin can be a thrombin receptor binding domain or a portion thereof. In one embodiment, the thrombin receptor binding domain or portion thereof comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). Another portion of a thrombin receptor binding domain comprises the polypeptide Glu-Gly-Lys-Arg-Gly-Asp-Ala-Cys-Glu-Gly (SEQ ID NO:7).

### BRIEF DESCRIPTION OF THE DRAWINGS

This invention will be further described with reference being made to the drawings in which:

FIG. 1A illustrates a bar graph representing results of the experiment of Example 1 in which KML4A tumor cells were added to a monolayer of human microvascular endothelial cells and assayed for the number of cells that bind to the endothelial cells and migrate through the endothelial layer.

FIG. 1B illustrates an assay diagram that demonstrates some of the steps of the assay used to estimate the metastatic potential of the tumor cells to migrate out of a blood vessel into a new tissue site.

FIG. 2 illustrates a bar graph representing results of the experiment of Example 2 to test the potential of TP508 to prevent transedothelial migration of metastatic colon cancer cells (KML4A) in both control cells and those activated with thrombin.

FIG. 3 illustrates a bar graph representing results of the experiment of Example 3 to test the effect of TP508 on binding of metastatic colon cancer cells (KML4A) to untreated and TNF alpha-activated endothelial cell surfaces.

FIG. 4 illustrates a series of pictures showing phase contrast micrographs of human microvascular endothelial cells following co-incubation with KLM4A colon cancer cells.

FIG. 5 illustrates two pictures showing phase contrast micrographs of human umbilical vein endothelial cells following co-incubation with KLM4A colon cancer cells.

FIG. 6 illustrates an effect of TNF alpha (T), TP508 (TP), saline (C), or combination of TNF alpha and TP508 (T+TP) on expression of mRNA for the protein known as “metastasis associated in colon cancer 1 (MACC1).”

FIG. 7 illustrates an effect of TNF alpha (T), TP508 (TP), saline (C), or combination of TNF alpha and TP508 (T+TP) on expression of melanoma cell adhesion molecule (MCAM).

FIG. 8 illustrates an effect of TNF alpha (T), TP508 (TP), saline (C), or combination of TNF alpha and TP508 (T+TP) on expression of snail homolog 3 (SNAI3).

FIG. 9 illustrates an effect of TNF alpha (TN), TP508 (TP), saline (C), or combination of TNF alpha and TP508 (T+TP) on expression of bone morphogenetic protein 9 (BMP9).

### DETAILED DESCRIPTION OF THE INVENTION

The invention includes methods of treating cancer in a subject, for example a human patient, comprising administering to the subject a therapeutically effective amount of an agonist of a non-proteolytically activated thrombin receptor (hereinafter “NPAR”) or a pharmaceutically acceptable salt thereof. In one embodiment, the NPAR agonist (e.g., thrombin derivative peptide) reduces or inhibits proliferation or metastasis of cancer. The disclosed NPAR agonists (e.g., thrombin derivative peptide) can reduce the penetration or movement of cancer cells through the endothelial lining of vessels. Because metastatic cancer cells can disperse throughout the body by adhering to the endothelial cell lining of the vessel before transmigrating across the endothelial lining and basement membrane barrier and ultimately invading the surrounding stroma to form new microcolonies, reducing penetration of the endothelial lining is one mechanism by which the disclosed NPAR agonists can reduce metastasis.

As used herein, “cancer cell” refers to a cell which exhibits the properties of uncontrolled growth or cell division, invasion and metastasis.

As used herein, “metastasis” refers to a process by which a cancer cell spreads from its site of origin to another site, which may be in another tissue or organ. Metastatic cancer cells can dissociate from a cancerous tissue, infiltrate through blood vessels into the blood circulation and migrate to a second site in the same tissue or organ or to other parts of the body. Metastatic cancer cells can also disperse throughout the body by infiltrating into lymph vessels, circulating with the lymphatic fluid, invading the lymphatic circulation glands, and eventually entering into the blood vessel. The capacity to metastasize is a characteristic of all malignant tumors. Metastatic cancer cells can disperse throughout the body by adhering to the endothelial cell lining of the vessel before transmigrating across the endothelial lining and basement membrane barrier. Ultimately, they can invade the surrounding stroma to form new microcolonies.

As used to herein, “cancer” includes, but not limited to, bladder cancer, breast cancer, colon cancer, rectal cancer, endometrial cancer, kidney (renal) cancer, liver cancer, leukemia, lung cancer, pancreatic cancer, prostate cancer, non-melanoma skin cancer, melanoma, thyroid cancer, stomach cancer, lymphoma, non-Hodgkin lymphoma, burkitt lymphoma, Kaposi sarcoma, basal cell carcinoma, skin cancer, bile duct cancer, gallbladder cancer, osteosarcoma, malignant fibrous histiocytoma, brain stem glioma, salivary gland cancer, brain cancer, bronchial cancer, extrahepatic bile duct cancer, cerebellar astrocytoma, ependymoblastoma, ependymoma, hypothalamic glioma, esophageal cancer, eye cancer, laryngeal cancer, ovarian cancer, cervical cancer, vaginal cancer, and testicular cancer.

As used herein, “treating” or “treatment” of cancer in a patient refers to 1) inhibiting the disease or arresting its development; 2) ameliorating or causing regression of the disease; 3) reducing the growth of the tumors; 4) reducing the spread of cancer, i.e., reducing metastasis; 5) reducing the likelihood of metastasis of the cancer; 6) reducing the penetration or movement of cancer cells through the endothelial lining of vessels; and/or 6) reducing the spread and/or growth of tumors, e.g., at least 5%, 10%, 5%. 20%, 30% reduction in the size and/or numbers of tumors relative to untreated controls.

The NPAR agonist can be administered any time during cancer growth and proliferation by cancer cells. Preferably, the NPAR agonist is administered any time before or during metastasis.

Compounds which stimulate an NPAR are said to be NPAR agonists. One such NPAR is a high-affinity thrombin receptor present on the surface of most cells. This NPAR component is largely responsible for high-affinity binding of thrombin, proteolytically inactivated thrombin, and thrombin derived peptides to cells. This NPAR appears to mediate a number of cellular signals that are initiated by thrombin independent of its proteolytic activity (see Sower, et. al., Experimental Cell Research, 247:422 (1999)). This NPAR is therefore characterized by its high affinity interaction with thrombin at cell surfaces and its activation by proteolytically inactive derivatives of thrombin and thrombin derived peptide agonists as described below. NPAR activation can be assayed based on the ability of molecules to stimulate cell proliferation when added to fibroblasts in the presence of submitogenic concentrations of thrombin or molecules that activate protein kinase C, as disclosed in U.S. Pat. Nos. 5,352,664 and 5,500,412. The entire teachings of these patents are incorporated herein by reference. NPAR agonists can be identified by this activation or by their ability to compete with ¹²⁵I-thrombin binding to cells.

A thrombin receptor binding domain is defined as a polypeptide or portion of a polypeptide which directly binds to the thrombin receptor and/or competitively inhibits binding between high-affinity thrombin receptors and alpha-thrombin.

NPAR agonists of the present invention include thrombin derivative peptides, modified thrombin derivative peptides and thrombin derivative peptide dimers as disclosed herein.

### Thrombin Peptide Derivative

Among NPAR agonists are thrombin peptide derivatives (also: “thrombin derivative peptides”), which are analogs of thrombin that have an amino acid sequence derived at least in part from that of thrombin and are active at a non-proteolytically activated thrombin receptor. Thrombin peptide derivatives include, for example, peptides that are produced by recombinant DNA methods, peptides produced by enzymatic digestion of thrombin, and peptides produced synthetically, which can comprise amino acid substitutions compared to thrombin, and/or modified amino acid residues, especially at the termini.

It is to be understood that all peptides described herein contain ionizable groups (i.e., the amino group of the N-terminal reside, the carboxyl group of the C-terminal residue and/or the amino acids in the side chains of the peptides). A person having ordinary skill in the art would understand that these ionizable groups contribute to the net charge of the thrombin peptide derivatives as referred to herein, in addition to the pH of the solution in which these peptides exist. As ionic substances which can be present in an acid or base form, the thrombin peptide derivatives as referred to herein can exist in various salt forms depending on their ionization state. Therefore, it is to be understood that when a thrombin peptide derivative is described herein by amino acid sequence or by some other description, corresponding pharmaceutically suitable salts thereof are also included.

NPAR agonists of the present invention include thrombin derivative peptides described in U.S. Pat. Nos. 5,352,664 and 5,500,412. In one embodiment, the NPAR agonist of the present invention is a thrombin peptide derivative or a physiologically functional equivalent, i.e., a polypeptide with no more than about fifty amino acid residues, preferably no more than about thirty amino acid residues and having sufficient homology to the fragment of human thrombin corresponding to thrombin amino acid residues 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val; SEQ ID NO:6) that the polypeptide activates NPAR. The thrombin peptide derivatives or modified thrombin peptide derivatives described herein preferably have from about 12 to about 23 amino acid residues, more preferably from about 19 to about 23 amino acid residues.

In another embodiment, the NPAR agonist of the present invention is a thrombin peptide derivative comprising a moiety represented by Structural Formula (I):

Asp-Ala-R (I).

R is a serine esterase conserved domain. Serine esterases (e.g., trypsin, thrombin, chymotrypsin and the like) have a region that is highly conserved. “Serine esterase conserved domain” refers to a polypeptide having the amino acid sequence of one of these conserved regions or is sufficiently homologous to one of these conserved regions such that the thrombin peptide derivative retains NPAR activating ability.

A physiologically functional equivalent of a thrombin derivative encompasses molecules which differ from thrombin derivatives in particulars which do not affect the function of the thrombin receptor binding domain or the serine esterase conserved amino acid sequence. Such particulars may include, but are not limited to, conservative amino acid substitutions and modifications, for example, amidation of the carboxyl terminus, acetylation of the amino terminus, conjugation of the polypeptide to a physiologically inert carrier molecule, or sequence alterations in accordance with the serine esterase conserved sequences.

A domain having a serine esterase conserved sequence can comprise a polypeptide sequence containing 4-12 of the N-terminal amino acid residues of the dodecapeptide previously shown to be highly conserved among serine proteases (Asp-X₁-Cys-X₂-Gly-Asp-Ser-Gly-Gly-Pro-X₃-Val; SEQ ID NO:13); wherein X₁ is either Ala or Ser; X₂ is either Glu or Gln; and X₃ is Phe, Met, Leu, His, or Val.

In one embodiment, the serine esterase conserved sequence comprises the amino acid sequence Cys-Glu-Gly-Asp-Ser-Gly-Gly-Pro-Phe-Val (SEQ ID NO:14) or a C-terminal truncated fragment of a polypeptide having the amino acid sequence of SEQ ID NO:14. It is understood, however, that zero, one, two or three amino acid residues in the serine esterase conserved sequence can differ from the corresponding amino acid in SEQ ID NO:14. Preferably, the amino acid residues in the serine esterase conserved sequence which differ from the corresponding amino acid in SEQ ID NO:14 are conservative substitutions, and are more preferably highly conservative substitutions. A “C-terminal truncated fragment” refers to a fragment remaining after removing an amino acid residue or block of amino acid residues from the C-terminus, said fragment having at least six and more preferably at least nine amino acid residues.

In another embodiment, the serine esterase conserved sequence comprises the amino acid sequence of SEQ ID NO:15 (Cys-X₁-Gly-Asp-Ser-Gly-Gly-Pro-X₂-Val; X₁ is Glu or Gln and X₂ is Phe, Met, Leu, His or Val) or a C-terminal truncated fragment thereof having at least six amino acid residues, preferably at least nine amino acid residues.

In a preferred embodiment, the thrombin peptide derivative comprises a serine esterase conserved sequence and a polypeptide having a more specific thrombin amino acid sequence Arg-Gly-Asp-Ala (SEQ ID NO:16). One example of a thrombin peptide derivative of this type comprises Arg-Gly-Asp-Ala-Cys-X₁-Gly-Asp-Ser-Gly-Gly-Pro-X₂-Val (SEQ ID NO:1). X₁ and X₂ are as defined above. The thrombin peptide derivative can comprise the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6), or an N-terminal truncated fragment thereof, provided that zero, one, two or three amino acid residues at positions 1-9 in the thrombin peptide derivative differ from the amino acid residue at the corresponding position of SEQ ID NO:6. Preferably, the amino acid residues in the thrombin peptide derivative which differ from the corresponding amino acid residues in SEQ ID NO:6 are conservative substitutions, and are more preferably highly conservative substitutions. An “N-terminal truncated fragment” refers to a fragment remaining after removing an amino acid residue or block of amino acid residues from the N-terminus, preferably a block of no more than six amino acid residues, more preferably a block of no more than three amino acid residues.

Optionally, the thrombin peptide derivatives described herein can be amidated at the C-terminus and/or acylated at the N-terminus. In a specific embodiment, the thrombin peptide derivatives comprise a C-terminal amide and optionally comprise an acylated N-terminus, wherein said C-terminal amide is represented by —C(O)NRₐR_{b}, wherein Rₐ and R_{b} are independently hydrogen, a substituted or unsubstituted aliphatic group comprising up to 10 carbon atoms, or Rₐ and R_{b}, taken together with the nitrogen to which they are bonded, form a C₃-C₁₀ non-aromatic heterocyclic group, and said N-terminal acyl group is represented by R_{c}C(O)—, wherein R_{c} is hydrogen, a substituted or unsubstituted aliphatic group comprising up to 10 carbon atoms, or a C₃-C₁₀ substituted or unsubstituted aromatic group. In another specific embodiment, the N-terminus of the thrombin peptide derivative is free (i.e., unsubstituted) and the C-terminus is free (i.e., unsubstituted) or amidated, preferably as a carboxamide (i.e., —C(O)NH₂). In a specific embodiment, the thrombin peptide derivative comprises the following amino acid sequence: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). In another specific embodiment, the thrombin peptide derivative comprises the amino sequence of Arg-Gly-Asp-Ala-Cys-Glu-Gly-Asp-Ser-Gly-Gly-Pro-Phe-Val (SEQ ID NO:17). Alternatively, the thrombin peptide derivative comprises the amino acid sequence of SEQ ID NO:18: Asp-Asn-Met-Phe-Cys-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe. The thrombin peptide derivatives comprising the amino acid sequences SEQ ID NO: 6, 17, or 18 can optionally be amidated at the C-terminus and/or acylated at the N-terminus. Preferably, the N-terminus is free (i.e., unsubstituted) and the C-terminus is free (i.e., unsubstituted) or amidated, preferably a carboxamide (i.e., —C(O)NH₂). It is understood, however, that zero, one, two or three amino acid residues at positions 1-9 and 14-23 in the thrombin peptide derivative can differ from the corresponding amino acid in SEQ ID NO:6. It is also understood that zero, one, two or three amino acid residues at positions 1-14 and 19-33 in the thrombin peptide derivative can differ from the corresponding amino acid in SEQ ID NO:18. Preferably, the amino acid residues in the thrombin peptide derivative which differ from the corresponding amino acid in SEQ ID NO:6 or SEQ ID NO:18 are conservative substitutions, and are more preferably highly conservative substitutions. Alternatively, an N-terminal truncated fragment of the thrombin peptide derivative having at least fourteen amino acid residues or a C-terminal truncated fragment of the thrombin peptide derivative having at least eighteen amino acid residues is a thrombin peptide derivative to be used as an NPAR agonist.

A “C-terminal truncated fragment” refers to a fragment remaining after removing an amino acid or block of amino acid residues from the C-terminus. An “N-terminal truncated fragment” refers to a fragment remaining after removing an amino acid residue or block of amino acid residues from the N-terminus. It is to be understood that both C-terminal truncated fragments and N-terminal truncated fragments can optionally be amidated at the C-terminus and/or acylated at the N-terminus.

A preferred thrombin peptide derivative for use in the disclosed methods comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2). Another preferred thrombin peptide derivative for use in the disclosed method comprises the polypeptide Asp-Asn-Met-Phe-Cys-Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe (SEQ ID NO:19). X₁ is Glu or Gln; X₂ is Phe, Met, Leu, His or Val. The thrombin peptide derivatives of SEQ ID NO:2 and SEQ ID NO:19 can optionally comprise a C-terminal amide and/or acylated N-terminus, as defined above. Preferably, the N-terminus is free (i.e., unsubstituted) and the C-terminus is free (i.e., unsubstituted) or amidated, preferably as a carboxamide (i.e., —C(O)NH₂). Alternatively, N-terminal truncated fragments of these preferred thrombin peptide derivatives, the N-terminal truncated fragments having at least fourteen amino acid residues, or C-terminal truncated fragments of these preferred thrombin peptide derivatives, the C-terminal truncated fragments having at least eighteen amino acid residues, can also be used in the disclosed methods.

TP508 is an example of a thrombin peptide derivative and is 23 amino acid residues long, wherein the N-terminal amino acid residue Ala is unsubstituted and the COOH of the C-terminal amino acid Val is modified to an amide represented by —C(O)NH₂ (SEQ ID NO:3). Another example of a thrombin peptide derivative comprises the amino acid sequence of SEQ ID NO:6, wherein both N- and C-termini are unsubstituted (“deamide TP508”). Other examples of thrombin peptide derivatives which can be used in the disclosed method include N-terminal truncated fragments of TP508 (or deamide TP508), the N-terminal truncated fragments having at least fourteen amino acid residues, or C-terminal truncated fragments of TP508 (or deamide TP508), the C-terminal truncated fragments having at least eighteen amino acid residues.

As used herein, a “conservative substitution” in a polypeptide is the replacement of an amino acid with another amino acid that has the same net electronic charge and approximately the same size and shape. Amino acid residues with aliphatic or substituted aliphatic amino acid side chains have approximately the same size when the total number of carbon and heteroatoms in their side chains differs by no more than about four. They have approximately the same shape when the number of branches in their side chains differs by no more than one. Amino acid residues with phenyl or substituted phenyl groups in their side chains are considered to have about the same size and shape. Listed below are five groups of amino acids. Replacing an amino acid residue in a polypeptide with another amino acid residue from the same group results in a conservative substitution:
Group I: glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).
Group II: glutamic acid, aspartic acid and non-naturally occurring amino acids with carboxylic acid substituted C1-C4 aliphatic side chains (unbranched or one branch point).
Group III: lysine, ornithine, arginine and non-naturally occurring amino acids with amine or guanidino substituted C1-C4 aliphatic side chains (unbranched or one branch point).
Group IV: glutamine, asparagine and non-naturally occurring amino acids with amide substituted C1-C4 aliphatic side chains (unbranched or one branch point).
Group V: phenylalanine, phenylglycine, tyrosine and tryptophan.

As used herein, a “highly conservative substitution” in a polypeptide is the replacement of an amino acid with another amino acid that has the same functional group in the side chain and nearly the same size and shape. Amino acids with aliphatic or substituted aliphatic amino acid side chains have nearly the same size when the total number of carbon and heteroatoms in their side chains differs by no more than two. They have nearly the same shape when they have the same number of branches in their side chains. Examples of highly conservative substitutions include valine for leucine, threonine for serine, aspartic acid for glutamic acid and phenylglycine for phenylalanine Examples of substitutions which are not highly conservative include alanine for valine, alanine for serine and aspartic acid for serine.

### Modified Thrombin Peptide Derivatives

In one embodiment of the invention, the NPAR agonists are modified relative to the thrombin peptide derivatives described above, wherein cysteine residues of aforementioned thrombin peptide derivatives are replaced with amino acids having similar size and charge properties to minimize dimerization of the peptides. Examples of suitable amino acids include alanine, glycine, serine, and an S-protected cysteine. Preferably, cysteine is replaced with alanine or serine. The modified thrombin peptide derivatives have about the same biological activity as the unmodified thrombin peptide derivatives.

It will be understood that the modified thrombin peptide derivatives disclosed herein can optionally comprise C-terminal amides and/or N-terminal acyl groups, as described above. Preferably, the N-terminus of a thrombin peptide derivative is free (i.e., unsubstituted) and the C-terminus is free (i.e., unsubstituted) or amidated, preferably as a carboxamide (i.e., —C(O)NH₂).

In a specific embodiment, the modified thrombin peptide derivative comprises a polypeptide Arg-Gly-Asp-Ala-Xaa-X₁-Gly-Asp-Ser-Gly-Gly-Pro-X₂-Val (SEQ ID NO:4), or a C-terminal truncated fragment thereof having at least six amino acids. More specifically, the thrombin peptide derivative comprises the polypeptide Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val (SEQ ID NO:20), or a fragment thereof comprising amino acid residues 10-18 of SEQ ID NO:20. Even more specifically, the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5), or a fragment thereof comprising amino acid residues 10-18 of SEQ ID NO:5. Xaa is alanine, glycine, serine or an S-protected cysteine. X₁ is Glu or Gln and X₂ is Phe, Met, Leu, His or Val. In one embodiment, X₁ is Glu, X₂ is Phe, and Xaa is Ala. In another embodiment, X₁ is Glu, X₂ is Phe, and Xaa is Ser. One example of a thrombin peptide derivative of this type is the polypeptide Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val (SEQ ID NO:21). A further example of a thrombin peptide derivative of this type is the polypeptide H-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-NH₂ (SEQ ID NO:22), wherein His a hydrogen atom of alanine indicating no modification at the N-terminus, and NH₂ indicates amidation at the C-terminus as —C(O)NH₂. Zero, one, two or three amino acids in the thrombin peptide derivative differ from the amino acid at the corresponding position of SEQ ID NO:4, 20, 5, 21 or 22, provided that Xaa is alanine, glycine, serine and an S-protected cysteine. Preferably, the difference is conservative.

In another specific embodiment, the thrombin peptide derivative comprises the polypeptide Asp-Asn-Met-Phe-Xbb-Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val-Met-Lys-Ser-Pro-Phe (SEQ ID NO:23), or a fragment thereof comprising amino acids 6-28. More preferably, the thrombin peptide derivative comprises the polypeptide Asp-Asn-Met-Phe-Xbb-Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val-Met-Lys-Ser-Pro-Phe (SEQ ID NO:24), or a fragment thereof comprising amino acids 6-28. Xaa and Xbb are independently alanine, glycine, serine or an S-protected cysteine. X₁ is Glu or Gln and X₂ is Phe, Met, Leu, His or Val. Preferably X₁ is Glu, X₂ is Phe, and Xaa and Xbb are alanine. One example of a thrombin peptide derivative of this type is a polypeptide comprising the amino acid sequence Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe (SEQ ID NO:25). A further example of a thrombin peptide derivative of this type is the polypeptide H-Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe-NH₂ (SEQ ID NO:26), wherein His a hydrogen atom of aspartic acid indicating no modification at the N-terminus, and NH₂ indicates amidation at the C-terminus as —C(O)NH₂. Zero, one, two or three amino acids in the thrombin peptide derivative can differ from the amino acid at the corresponding position of SEQ ID NO:23, 24, 25 or 26. Xaa and Xbb are independently alanine, glycine, serine or an S-protected cysteine. Preferably, the difference is conservative.

An “S-protected cysteine” is a cysteine residue in which the reactivity of the thiol moiety, —SH, is blocked with a protecting group. Suitable protecting groups are known in the art and are disclosed, for example, in T. W. Greene and P. G. M. Wuts, Protective Groups in Organic Synthesis, 3^{rd} Edition, John Wiley & Sons, (1999), pp. 454-493, the teachings of which are incorporated herein by reference in their entirety. Suitable protecting groups should be non-toxic, stable in pharmaceutical formulations and have minimum additional functionality to maintain the activity of the thrombin peptide derivative. A free thiol can be protected as a thioether, a thioester, or can be oxidized to an unsymmetrical disulfide. Preferably the thiol is protected as a thioether. Suitable thioethers include, but are not limited to, S-alkyl thioethers (e.g., C₁-C₅ alkyl), and S-benzyl thioethers (e.g., cysteine-S—S-t-Bu). Preferably the protective group is an alkyl thioether. More preferably, the S-protected cysteine is an S-methyl cysteine. Alternatively, the protecting group can be: 1) a cysteine or a cysteine-containing peptide (the “protecting peptide”) attached to the cysteine thiol group of the thrombin peptide derivative by a disulfide bond; or 2) an amino acid or peptide (“protecting peptide”) attached by a thioamide bond between the cysteine thiol group of the thrombin peptide derivative and a carboxylic acid in the protecting peptide (e.g., at the C-terminus or side chain of aspartic acid or glutamic acid). The protecting peptide can be physiologically inert (e.g., a polyglycine or polyalanine of no more than about fifty amino acids optionally interrupted by a cysteine), or can have a desirable biological activity.

The thrombin peptide derivatives or the modified thrombin peptide derivatives of the present invention can be mixed with a dimerization inhibitor for the preparation of a pharmaceutical composition comprising the thrombin peptide derivatives or the modified thrombin peptide derivatives of the present invention. Dimerization inhibitors can include chelating agents and/or thiol-containing compounds. An antioxidant can also be used in combination with the chelating agent and/or the thiol-containing compound.

A “chelating agent,” as used herein, is a compound having multiple sites (two, three, four or more) which can simultaneously bind to a metal ion or metal ions such as, for example, lead, cobalt, iron or copper ions. The binding sites typically comprise oxygen, nitrogen, sulfur or phosphorus. For example, salts of EDTA (ethylenediaminetetraacetic acid) can form at least four to six bonds with a metal ion or metal ions via the oxygen atoms of four acetic acid moieties (—CH₂C(O)O⁻) and the nitrogen atoms of ethylenediamine moieties (>N—CH₂—CH₂—N<) of EDTA. It is understood that a chelating agent also includes a polymer which has multiple binding sites to a metal or metal ions. Preferably, a chelating agent of the invention is non-toxic and does not cause unacceptable side effects at the dosages of pharmaceutical composition being administered in the methods of the invention. As a chelating agent of the invention, a copper-chelating agent is preferable. A “copper-chelating agent” refers to a chelating agent which can bind to a copper ion or copper ions. Examples of the copper-chelating agent include ethylenediaminetetraacetic acid (EDTA), penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine (TEPA), triethylenetetraamine and tris(2-carboxyethyl)phosphine (TCEP). Additional chelating agents are diethylenetriaminepentacetic acid (DTPA) and bathophenanthroline disulfonic acid (BPADA). EDTA is a preferred chelating agent. Typical amounts of a chelating agent present in the pharmaceutical compositions of the instant invention are in a range of between about 0.00001% and about 0.1% by weight, preferably between about 0.0001% and about 0.05% by weight.

A “pharmaceutically acceptable thiol-containing compound” as used herein is a compound which comprises at least one thiol (—SH) group and which does not cause unacceptable side effects at the dosages which are being administered. Examples of a pharmaceutically acceptable thiol-containing compound include thioglycerol, mercaptoethanol, thioglycol, thiodiglycol, cysteine, thioglucose, dithiothreitol (DTT) and dithio-bis-maleimidoethane (DTME). Typically, between about 0.001% and about 5% by weight, preferably between about 0.05% and about 1.0% by weight of a pharmaceutically acceptable thiol-containing compound is present in the pharmaceutical compositions of the invention.

An “antioxidant,” as used herein, is a compound which is used to reduce an oxidation reaction caused by an oxidizing agent such as oxygen. Examples of antioxidants include tocopherol, cystine, methionine, glutathione, tocotrienol, dimethyl glycine, betaine, butylated hydroxyanisole, butylated hydroxytoluene, vitamin E, ascorbic acid, ascorbyl palmitate, thioglycolic acid and antioxidant peptides such as, for example, turmerin. Typically, between about 0.001% and about 10% by weight, preferably between about 0.01% and about 5%, more preferably between about 0.05% and about 2.0% by weight of an antioxidant is present in the pharmaceutical compositions of the invention.

It is understood that certain chelating agents or thiol-containing compounds may also function as antioxidants, for example, tris(2-carboxyethyl)phosphine, cysteine or dithiothreitol. Other types of commonly used antioxidants, however, do not contain a thiol group. It is also understood that certain thiol-containing compounds may also function as a chelating agent, for example, dithiothreitol. Other types of commonly used chelating agents, however, do not contain a thiol group. It is also understood that the pharmaceutical compositions of the instant invention can comprise more than one chelating agent, thiol-containing compound or antioxidant. That is, for example, a chelating agent can be used either alone or in combination with one or more other suitable chelating agents.

### Thrombin Peptide Derivative Dimers

In some aspects of the present invention, the NPAR agonists of the methods are thrombin peptide derivative dimers. The dimers essentially do not revert to monomers and still have about the same biological activity as the thrombin peptide derivative monomers described above. A “thrombin peptide derivative dimer” is a molecule comprising two thrombin peptide derivatives (polypeptides) linked by a covalent bond, preferably a disulfide bond between cysteine residues. Thrombin peptide derivative dimers are typically essentially free of the corresponding monomer, e.g., greater than 95% free by weight and preferably greater than 99% free by weight. Preferably the polypeptides are the same and covalently linked through a disulfide bond.

The thrombin peptide derivative dimers of the present invention comprise the thrombin peptide derivatives described above. Specifically, thrombin peptide derivatives have fewer than about fifty amino acids, preferably about thirty-three or fewer amino acids. The thrombin peptide derivative dimers described herein are formed from polypeptides typically having at least six amino acids and preferably from about 12 to about 33 amino acid residues, and more preferably from about 12 to about 23 amino acid residues. Thrombin peptide derivative monomer subunits of the dimers have sufficient homology to the fragment of human thrombin corresponding to thrombin amino acid residues 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6)) so that NPAR is activated.

In a specific embodiment, each of the two thrombin peptide derivatives (monomers) of a dimer comprises the polypeptide Arg-Gly-Asp-Ala-Cys-X₁-Gly-Asp-Ser-Gly-Gly-Pro-X₂-Val (SEQ ID NO:1), or a C-terminal truncated fragment thereof comprising at least six amino acid residues. More specifically, a polypeptide monomer comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6), or a fragment thereof comprising amino acid residues 10-18 of SEQ ID NO: 5. Even more specifically, a polypeptide monomer comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), or a fragment thereof comprising amino acid residues 10-18 of SEQ ID NO:2. X₁ is Glu or Gln and X₂ is Phe, Met, Leu, His or Val. Preferably X₁ is Glu, and X₂ is Phe. One example of a polypeptide of this type is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). A further example is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3), wherein H signifies a hydrogen atom of alanine indicating no modification at the N-terminus, and NH₂ signifies amidation at the C-terminus as —C(O)NH₂. Zero, one, two or three amino acid residues in the polypeptide differ from the amino acid residue at the corresponding position of SEQ ID NO:6, 1, 2, or 3. Preferably, the difference is conservative.

One example of a thrombin peptide derivative dimer of the present invention is represented by Formula (IV):

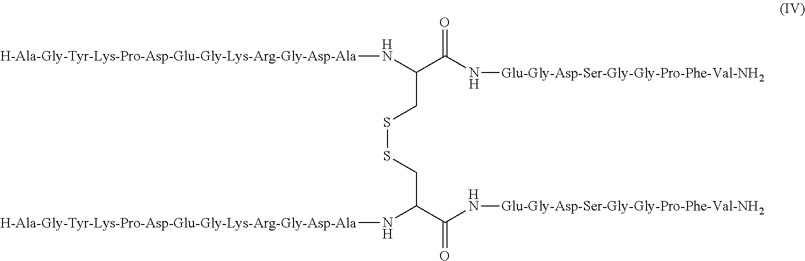

Thrombin peptide dimers are preferably substantially free of the corresponding monomer(s), e.g., at 95%, 98% or 99% by weight free of monomer(s).

In another specific embodiment, each of the two thrombin peptide derivatives (monomers) of a dimer comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27), or a C-terminal truncated fragment thereof having at least twenty-three amino acid residues. More preferably, a polypeptide comprises Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe-Asn-Asn-Arg-Trp-Tyr (SEQ ID NO:8), or a C-terminal truncated fragment thereof comprising at least twenty-three amino acid residues. X₁ is Glu or Gln and X₂ is Phe, Met, Leu, His or Val. Preferably X₁ is Glu, and X₂ is Phe. One example of a polypeptide of this type is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27). A further example of a polypeptide of this type is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr-NH₂ (SEQ ID NO:9), wherein H signifies a hydrogen atom of alanine indicating no modification at the N-terminus, and NH₂ indicates amidation at the C-terminus —C(O)NH₂. Zero, one, two or three amino acid residues in the polypeptide differ from the amino acid residue at the corresponding position of SEQ ID NO:27, 28 or 29. Preferably, the difference is conservative.

It is to be understood that all peptides described herein contain ionizable groups (i.e., the amino group of the N-terminal reside, the carboxyl group of the C-terminal residue and/or the amino acids in the side chains of the peptides). A person having ordinary skill in the art would understand that these ionizable groups contribute to the net charge of the thrombin peptide derivatives as referred to herein, in addition to the pH of the solution in which these peptides exist. As ionic substances which can be present in an acid or base form, the thrombin peptide derivatives as referred to herein can exist in various salt forms depending on their ionization state. Therefore, it is to be understood that when a thrombin peptide derivative is described herein by amino acid sequence or by some other description, corresponding pharmaceutically suitable salts thereof are also included.

Pharmaceutically acceptable salt forms include pharmaceutically acceptable acidic/anionic or basic/cationic salts. Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine and triethanolamine salts.

Methods of Treatment with NPAR Agonists

The present invention is directed to methods of treating cancer in a subject, comprising administering to the subject in need of treatment for cancer, a therapeutically effective amount of an NPAR agonist.

In some embodiments, there are provided methods of reducing or preventing metastasis of cancer in a subject in need thereof, the method comprising administering to the subject a therapeutically effective amount of an agonist of a non-proteolytically activated thrombin receptor.

In some embodiments, there are provided methods of treating colon cancer in a subject in need thereof, comprising administering to the subject a therapeutically effective amount of an NPAR agonist.

In some embodiments, there are provided methods of reducing or preventing metastasis of colon cancer in a subject in need thereof, comprising administering to the subject a therapeutically effective amount of an NPAR agonist.

In some embodiments, there are provided methods of reducing or preventing the likelihood of the metastasis of cancer in a subject in need thereof, comprising administering to the subject a therapeutically effective amount of an NPAR agonist.

In some embodiments, there are provided methods of reducing or inhibiting a penetration or movement of cancer cells through the endothelial lining in a subject in need thereof, comprising administering to the subject a therapeutically effective amount of an NPAR agonist.

In some embodiments, there are provided methods of reducing or inhibiting a penetration or movement of cancer cells through the endothelial lining of vessels thereby reducing or inhibiting the metastasis of cancer or reducing or preventing the likelihood of the metastasis of cancer in a subject in need thereof, comprising administering to the subject a therapeutically effective amount of an NPAR agonist.

In some embodiments, the methods provided herein are for colon cancer.

A thrombin peptide derivative can be tested for its effect on tumor growth, by implanting tumor cells at a site in laboratory animals, for example, in the lung or under the skin of the animals. The animals can be administered doses of the thrombin peptide derivative and tested over a period of time to assess whether the growth of the tumors has been inhibited or reduced, or whether the metastatic process has been inhibited or reduced, or the likelihood of the spread of the cancer or the likelihood of the metastasis of the cancer has been inhibited or reduced or the penetration or movement of cancer cells through the endothelial lining of vessels is inhibited or reduced, compared with a control group of laboratory animals treated with only saline or other appropriate vehicle used for administration of the thrombin derivative peptide. The thrombin derivative peptide is effective in inhibiting tumor growth or reducing or inhibiting the metastasis of tumor or reducing or inhibiting the penetration or movement of cancer cells through the endothelial lining of vessels if the animals treated with peptide have smaller tumors or fewer tumors than the animals treated with vehicle only, e.g., at least 5%, 10%, 5%. 20%, 30% reduction in the size and/or numbers of tumors relative to untreated controls.

The ability of a thrombin peptide derivative to inhibit metastasis can be tested by injecting or implanting tumor cells known to metastasize, (e.g., melanoma cells, colon tumor cells etc.) into laboratory animals, (e.g., injecting into the tail vein of rats). A thrombin derivative peptide can be administered to one group of animals, and a control group of animals can be treated with only saline or other appropriate vehicle used for administration of the thrombin derivative peptide. Following treatment, the animals can be studied for metastases. The thrombin derivative peptide is effective in inhibiting metastasis if the peptide-treated animals have fewer metastases than the control group.

One of ordinary skill in the art is able to devise more detailed protocols for these experiments, and is familiar with other procedures available to test the effectiveness of thrombin derivative peptides in the claimed methods.

The disclosed methods of cancer treatment can be used as a primary treatment method during the cancer progression and proliferation including metastasis, i.e., as the only anticancer agent being used to treat the patient (monotherapy). Alternatively, the agonist of a non-proteolytically activated thrombin receptor is administered in combination with a therapeutically effective amount of another anticancer agent, e.g., Anastrozole (Arimidex®), Azacitidine (Vidaza®), Bevacizumab (Avastin™), Bicalutamide, Casodex®), Bortezomib (Velcade®), Capecitabine (Xeloda®), Carboplatin (Paraplatin®), Cetuximab (Erbitux™), Dasatinib (Sprycel™), Docetaxel (Taxotere®), Doxorubicin Liposomal, (Doxil®Caelyx®), Epirubicin (Ellence®), Erlotinib (Tarceva®), Exemestane (Aromasin®), Gefitinib (Iressa®), Gemcitabine (Gemzar®), Goserelin (Zoladex®), Imatinib, STI-571 (Gleevec®), Irinotecan (Camptosar®), Lapatinib (Tykerb), Letrozole (Femara®), Oxaliplatin (Eloxatin™), Paclitaxel (Onxol™Paxene®Taxol®), Pemetrexed (Alimta®), Rituximab (Rituxan®), Sorafenib (Nexavar®), Sunitinib (Sutent®), Tamoxifen (Nolvadex®), Temozolomide (Temodar®), Trastuzumab (Herceptin®), Triptorelin (Trelstar™ Depot) or Vinorelbine (Navelbine®).

The disclosed methods are not limited to any particular kind of cancer or tissue or location of the body. Examples of cancer which can be treated by the disclosed methods include, but, are not limited to, bladder cancer, breast cancer, colon and rectal cancer, endometrial cancer, kidney (renal) cancer, liver cancer, leukemia, lung cancer, pancreatic cancer, prostate cancer, non-melanoma skin cancer, melanoma, thyroid cancer, stomach cancer, lymphoma, non-Hodgkin lymphoma, Kaposi sarcoma, basal cell carcinoma, skin cancer, bile duct cancer, gallbladder cancer, osteosarcoma, malignant fibrous histiocytoma, brain stem glioma, salivary gland cancer, brain cancer, burkitt lymphoma, bronchial cancer, extrahepatic bile duct cancer, cerebellar astrocytoma, ependymoblastoma, ependymoma, hypothalamic glioma esophageal cancer, eye cancer, laryngeal cancer, cervical cancer, ovarian cancer, vaginal cancer, and testicular cancer.

In some embodiments, cancers that can be treated or prevented by the methods of the present invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, ovarian cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

Other examples of leukemias include acute and/or chronic leukemias, e.g., lymphocytic leukemia (e.g., as exemplified by the p388 (murine) cell line), large granular lymphocytic leukemia, and lymphoblastic leukemia; T-cell leukemias, e.g., T-cell leukemia (e.g., as exemplified by the CEM, Jurkat, and HSB-2 (acute), YAC-1 (murine) cell lines), T-lymphocytic leukemia, and T-lymphoblastic leukemia; B cell leukemia (e.g., as exemplified by the SB (acute) cell line), and B-lymphocytic leukemia; mixed cell leukemias, e.g., B and T cell leukemia and B and T lymphocytic leukemia; myeloid leukemias, e.g., granulocytic leukemia, myelocytic leukemia (e.g., as exemplified by the HL-60 (promyelocyte) cell line), and myelogenous leukemia (e.g., as exemplified by the K562 (chronic) cell line); neutrophilic leukemia; eosinophilic leukemia; monocytic leukemia (e.g., as exemplified by the THP-1 (acute) cell line); myelomonocytic leukemia; Naegeli-type myeloid leukemia; and nonlymphocytic leukemia. Other examples of leukemias are described in Chapter 60 of The Chemotherapy Sourcebook, Michael C. Perry Ed., Williams & Williams (1992) and Section 36 of Holland Frie Cancer Medicine 5th Ed., Bast et al. Eds., B.C. Decker Inc. (2000). The entire teachings of the preceding references are incorporated herein by reference.

In one embodiment, the disclosed method is believed to be effective in treating a subject with non-solid tumors such as multiple myeloma. In another embodiment, the disclosed method is believed to be effective against T-leukemia (e.g., as exemplified by Jurkat and CEM cell lines); B-leukemia (e.g., as exemplified by the SB cell line); promyelocytes (e.g., as exemplified by the HL-60 cell line); uterine sarcoma (e.g., as exemplified by the MES-SA cell line); monocytic leukemia (e.g., as exemplified by the THP-1 (acute) cell line); and lymphoma (e.g., as exemplified by the U937 cell line).

A “subject” is preferably a human, but can also be an animal in need of treatment with a thrombin receptor agonist, e.g., companion animals (e.g., dogs, cats, and the like), farm animals (e.g., cows, pigs, horses and the like) and laboratory animals (e.g., rats, mice, guinea pigs and the like).

A “therapeutically effective amount” is the quantity of the NPAR agonist that results in a reduced level or inhibition of metastasis or cancer proliferation or results in reducing the likelihood of the metastasis of cancer or results in reducing or inhibiting the penetration of the cancer cells through the endothelial lining of the vessel compared to untreated or sham-treated controls. NPAR agonists can effectively inhibit or reduce the extent of metastasis, growth, and/or progression of cancer. A therapeutically effective amount can be a quantity of NPAR agonist that results in a reduced extent of metastasis, growth, and/or progression of cancer and/or reduced penetration of the cancer cells through the endothelial lining of the vessel, as compared to untreated or sham-treated controls. The amount of the NPAR agonist administered will depend on the degree of severity of cancer, and the release characteristics of the pharmaceutical formulation. It will also depend on the subject's health, size, weight, age, sex and tolerance to drugs. When administered more than once, the NPAR agonists are preferably administered at evenly spaced intervals; each dose can be the same or different, but is preferably the same. A dose delivered to a subject can be, for example, 0.1-500 μg, preferably 1-50 μg of NPAR agonist, and is commonly 3, 5, 10, 30 or 50 μg per day.

In some embodiments, a dose delivered to a subject of NPAR agonist is about 0.5 mg/kg/day; or 1 mg/kg/day; or 1.5 mg/kg/day; or 2 mg/kg/day; or 2.5 mg/kg/day; or 3 mg/kg/day; or 3.5 mg/kg/day; or 4 mg/kg/day; or 4.5 mg/kg/day; or 5 mg/kg/day; or 5.5 mg/kg/day; or 6 mg/kg/day; or 6.5 mg/kg/day; or 7 mg/kg/day; or 7.5 mg/kg/day; or 8 mg/kg/day; or 8.5 mg/kg/day; or 9 mg/kg/day; or 9.5 mg/kg/day; or 10 mg/kg/day.

In some embodiments, a dose delivered to a subject of NPAR agonist is in a range of about 0.5-5 mg/kg/day; or 5-10 mg/kg/day; or 0.5-2 mg/kg/day; or 3-5 mg/kg/day; or 8-10 mg/kg/day; or 7-9 mg/kg/day.

The disclosed NPAR agonists can be administered by any suitable route, including, for example intravenous injection. The NPAR agonist can be administered locally by introduction to a cancer tissue. The NPAR agonist can be administered to the subject in a sustained release formulation, or can be delivered by a pump or an implantable device, or by an implantable carrier such as the polymers discussed below. “Administered to the cancer tissue” means delivered to the cancerous tumor from without or from within the tumor. Alternatively, the point of delivery of the NPAR agonist can be in sufficient proximity to cancer cells or surfaces of the cancerous tissue so that the agonist can diffuse and contact the cancerous tissue as well as migrate into and out of blood and lymph vessels.

The NPAR agonists can be administered to the subject in conjunction with an acceptable pharmaceutical carrier as part of a pharmaceutical composition. The formulation of the pharmaceutical composition will vary according to the mode of administration selected. Suitable pharmaceutical carriers may contain inert ingredients which do not interact with the NPAR agonist. The carriers should be biocompatible, i.e., non-toxic, non-inflammatory, non-immunogenic and devoid of other undesired reactions at the administration site. Examples of pharmaceutically acceptable carriers include, for example, saline, commercially available inert gels, or liquids supplemented with albumin, methyl cellulose or a collagen matrix. Further examples include sterile water, physiological saline, bacteriostatic saline (saline containing about 0.9% mg/ml benzyl alcohol), phosphate-buffered saline, Hank's solution, Ringer's-lactate and the like. Standard pharmaceutical formulation techniques can be employed, such as those described in Remington's Pharmaceutical Sciences (Remington's Pharmaceutical Sciences. XVIII, Mack Publishing Company, Easton, Pa. (1990)).

Pharmaceutical compositions may include gels. Gels are compositions comprising a base selected from an oleaginous base, water, or an emulsion-suspension base. To the base is added a gelling agent which forms a matrix in the base, increasing its viscosity to a semisolid consistency. Examples of gelling agents are hydroxypropyl cellulose, acrylic acid polymers, and the like. The active ingredients are added to the formulation at the desired concentration at a point preceding addition of the gelling agent or can be mixed after the gelation process.

In one embodiment, the NPAR agonists are administered in a sustained release formulation. Polymers are often used to form sustained release formulations. Examples of these polymers include poly α-hydroxy esters such as polylactic acid/polyglycolic acid homopolymers and copolymers, polyphosphazenes (PPHOS), polyanhydrides and poly (propylene fumarates).

Polylactic acid/polyglycolic acid (PLGA) homo and copolymers are well known in the art as sustained release vehicles. The rate of release can be adjusted by the skilled artisan by variation of polylactic acid to polyglycolic acid ratio and the molecular weight of the polymer (see Anderson, et al., Adv. Drug Deliv. Rev. 28:5 (1997), the entire teachings of which are incorporated herein by reference). The incorporation of poly-ethylene glycol into the polymer as a blend to form microparticle carriers allows further alteration of the release profile of the active ingredient (see Cleek et al., J. Control Release 48:259 (1997), the entire teachings of which are incorporated herein by reference). Ceramics such as calcium phosphate and hydroxyapatite can also be incorporated into the formulation to improve mechanical qualities.

PPHOS polymers contain alternating nitrogen and phosphorous with no carbon in the polymer backbone, as shown below in Structural Formula (II):

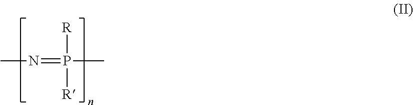

The properties of the polymer can be adjusted by suitable variation of side groups R and R′ that are bonded to the polymer backbone. For example, the degradation of and drug release by PPHOS can be controlled by varying the amount of hydrolytically unstable side groups. With greater incorporation of either imidazolyl or ethylglycol substituted PPHOS, for example, an increase in degradation rate is observed (see Laurencin et al., J Biomed Mater. Res. 27:963 (1993), the entire teachings of which are incorporated herein by reference), thereby increasing the rate of drug release.

Thrombin peptide derivatives and modified thrombin peptide derivatives can be synthesized by solid phase peptide synthesis (e.g., BOC or FMOC) method, by solution phase synthesis, or by other suitable techniques including combinations of the foregoing methods. The BOC and FMOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these six articles are incorporated herein by reference in their entirety.

Thrombin peptide derivative dimers can be prepared by oxidation of the monomer. Thrombin peptide derivative dimers can be prepared by reacting the thrombin peptide derivative with an excess of oxidizing agent. A well-known suitable oxidizing agent is iodine.

A “non-aromatic heterocyclic group” as used herein, is a non-aromatic carbocyclic ring system that has 3 to 10 atoms and includes at least one heteroatom, such as nitrogen, oxygen, or sulfur. Examples of non-aromatic heterocyclic groups include piperazinyl, piperidinyl, pyrrolidinyl, morpholinyl, thiomorpholinyl.

The term “aryl group” includes both carbocyclic and heterocyclic aromatic ring systems. Examples of aryl groups include phenyl, indolyl, furanyl and imidazolyl.

An “aliphatic group” is a straight chain, branched or cyclic non-aromatic hydrocarbon. An aliphatic group can be completely saturated or contain one or more units of unsaturation (e.g., double and/or triple bonds), but is preferably saturated, i.e., an alkyl group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. Aliphatic groups include, for example, methyl, ethyl, n-propyl, iso-propyl, n-butyl, sec-butyl, tert-butyl, pentyl, cyclopentyl, hexyl, cyclohexyl, octyl and cyclooctyl.

Suitable substituents for an aliphatic group, an aryl group or a non-aromatic heterocyclic group are those which do not significantly lower therapeutic activity of the NPAR agonist, for example, those found on naturally occurring amino acids. Examples include —OH, a halogen (—Br, —Cl, —I and —F), —O(Rₑ), —O—CO—(Rₑ), —CN, —NO₂, —COOH, ═O, —NH₂—NH(Rₑ), —N(Rₑ)₂. —COO(Rₑ), —CONH₂, —CONH(Rₑ), —CON(Rₑ)₂, —SH, —S(Rₑ), an aliphatic group, an aryl group and a non-aromatic heterocyclic group. Each Rₑ is independently an alkyl group or an aryl group. A substituted aliphatic group can have more than one substituent.

While this invention has been particularly shown and described with references to example embodiments thereof, it is understood by those skilled in the art that various changes in form and details may be made therein without departing from the scope of the invention encompassed by the appended claims.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

In the examples below as well as throughout the application, the following abbreviations have the following meanings. If not defined, the terms have their generally accepted meanings.

| | |
|---|---|
| BAEC = | bovine aortic endothelial cells |
| BMP9 = | bone morphogenetic protein 9 |
| CO₂ = | Carbon dioxide |
| cm = | centimeter |
| EBM = | endothelial cell basal medium |
| FBS = | fetal bovine serum |
| hr = | hours |
| MACC1 = | metastasis associated in colon cancer 1 |
| MCAM = | melanoma cell adhesion molecule |
| μg = | microgram |
| μl = | microliter |
| ml = | milliliter |
| ng = | nanogram |
| nm = | nanometer |
| SNAI3 = | snail homolog 3 |
| U/ml = | Units per milliliter |

### Example 1

### Effect of TP508 on the Migration of KML4a Tumor Cells Through the Endothelial Cells

In this experiment, metastatic colon tumor cells (KML4A) derived from a human colon cancer and selected for repeated metastasis to the liver (Morikawa et al. Cancer Research 48, 1943-1948, Apr. 1, 1988; and Morikawa et al. Cancer Research 48, 6863-6871, Dec. 1, 1988) were harvested from established cultures and labeled with fluorescent CytoTracker™ (Cell Biolabs, inc.) and re-established as a uniform cell suspension. Human endothelial cell monolayers were established in transmembrane inserts supplied by Cell Biolabs, Inc., by plating 50,000 human microvascular endothelial cells (Cambrex BioScience; Walkersville, Md.) per insert and culturing them in endothelial cell basal medium (EBM) with 2% fetal bovine serum (FBS) and growth factors (SingleQuots™, Clonetics, San Diego, Calif.) in 5% CO₂ at 37° C. for three days. The endothelial cell monolayers were then pretreated with saline (Control) or TP508 (50 μg/ml in saline) for 24 hours prior to addition of labeled KML4A cells (35,000 cells per insert in serum-free medium containing 1% bovine serum albumin). The inserts were then placed into wells of a 24-well tissue culture plate (BD Falcon) with 0.5 ml of growth medium containing 10% fetal bovine serum to act as a chemoatractant for the tumor cells. The number of KML4A cells exhibiting transendothelial migration was determined 18 hours later by measuring the amount of cell fluorescence recovered from the filter bottom using a SyNERGYHT-1 BioLabs Fluorescent Plate Reader (480/20 nm/528/20 nm). This data is expressed as relative fluorescence units.

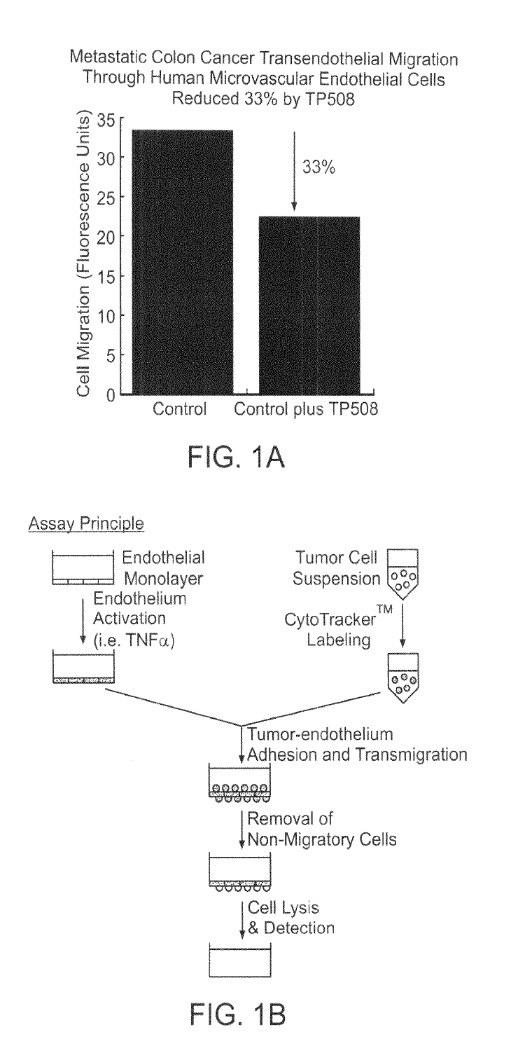

FIG. 1A is a bar graph representing results of the experiment in which KML4A tumor cells were added to a monolayer of human microvascular endothelial cells and assayed for the number of cells that bind to the endothelial cells and migrate through the endothelial layer. This data was obtained using the CytoSelect™ Transendothelial Migration Assay Kit (Cell Biolabs, Inc., San Diego, Calif.) using the protocol as described by the company. FIG. 1B is an assay diagram that demonstrates some of the steps of the assay used to estimate the metastatic potential of the tumor cells to migrate out of a blood vessel into a new tissue site by measuring their ability to penetrate or disrupt a monolayer of endothelial cells and migrate through the underlying membrane.

As illustrated in FIG. 1A, pretreatment of endothelial monolayers with TP508 decreased transendothelial migration by approximately thirty-three percent (33%).

### Example 2

### Effect of TP508 and/or Thrombin on the Migration of KML4a Tumor Cells Through the Endothelial Cells

In this experiment, as in Example 1, KML4A tumor cells derived from a human colon cancer and selected for repeated metastasis to the liver (Morikawa et al., supra) were harvested from established cultures and labeled with fluorescent CytoTracker™ (Cell Biolabs, inc.) and re-established as a uniform cell suspension. Human endothelial cell monolayers were established in transmembrane inserts supplied by Cell Biolabs, Inc., by plating 50,000 human umbilical vein endothelial cells (Cambrex BioScience; Walkersville, Md.) per insert and culturing them in endothelial cell basal medium (EBM) with 2% fetal bovine serum (FBS) and growth factors (SingleQuots™, Clonetics, San Diego, Calif.) in 5% CO₂ at 37° C. for three days. The endothelial cell monolayers were then pretreated with saline (Control) or TP508 (50 μg/ml in saline) followed by treatment with saline (control and control plus TP508) or thrombin (2 U/ml)(thrombin and thrombin plus TP508) for 24 hours. Fluorescently labeled KML4A tumor cells (35,000 cells per insert in serum-free medium containing 1% bovine serum albumin) were then added to the culture inserts and the inserts placed into wells of a 24-well tissue culture plate (BD Falcon) with 0.5 ml of growth medium containing 10% fetal bovine serum to act as a chemoatractant for the tumor cells. The number of cells migrating through the filter inserts to the filter bottom was determined 18 hr later by measuring the fluorescence of material recovered from the filter bottom using a SyQuest BioLabs Fluorescent Plate Reader (480 nm/529 nm). Data is expressed as Relative Fluorescence Units.

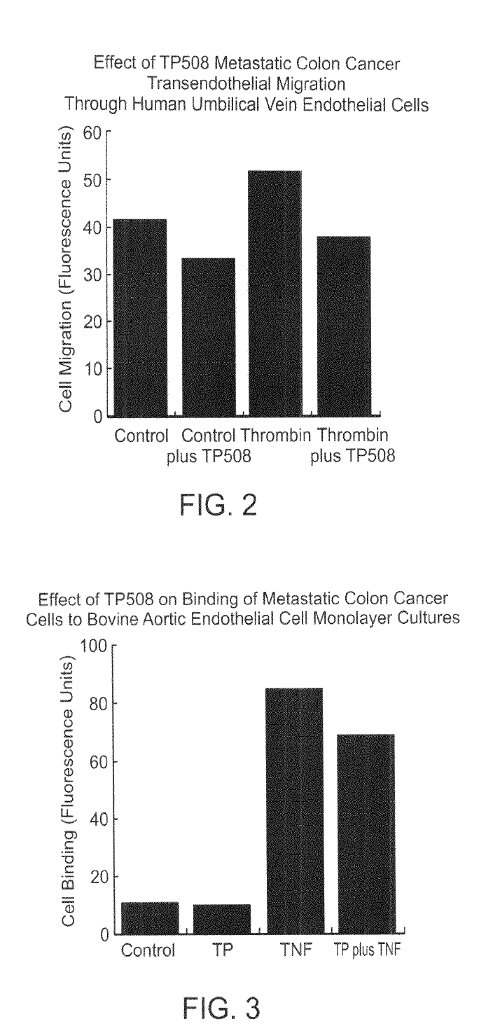

FIG. 2 is a bar graph representing results of the above experiment to test the potential of TP508 to prevent transedothelial migration of metastatic colon cancer cells (KML4A) in both control cells and those activated with thrombin. As shown in FIG. 2, TP508 decreased KML4A transedothelial migration by approximately 19 percent (19%) relative to untreated control cells. Thrombin, in contrast, increased transedothelial migration. TP508 pretreatment decreased or prevented the thrombin-induced increase in transedothelial migration resulting in a level of transedothelial migration in thrombin plus TP508 cultures similar to that seen in untreated control cells.

### Example 3

### Effect of TP508, TNF Alpha, and/or Thrombin on the Migration of KML4a Tumor Cells Through the Bovine Aortic Endothelial Cells

In this experiment, bovine aortic endothelial cells (BAEC; Cambrex BioScience; Walkersville, Md.) were plated in 24-well plastic tissue culture plates (BD Falcon™) at a density of 35,000 cells per cm² and cultured in endothelial cell basal medium (EBM) with 2% fetal bovine serum (FBS) and growth factors (SingleQuots™, Clonetics, San Diego, Calif.) in 5% CO₂ at 37° C. for three days. BAEC monolayer cultures were then pretreated with saline (Control) or TP508 (TP) and then treated with saline or TNF alpha (TNF, 50 ng/ml) for 24 hours. KML4A cells were labeled with fluorescent CytoTracker™ as described in Example 1 above and added to the BAEC monolayers in EBM medium complete with fetal bovine serum and SingleQuot growth factor supplements (30,000 cells per well). After thirty (30) minutes, the cell monlayers were rinsed two times to remove unbound KML4A cells and the monolayers were solubilized using 200 μl of Cell Solubilizing Solution (Cell Biolabs, Inc.). Fluorescence of the solubilized KML4A that had bound to the endothelial cells was measured by transferring aliquots of the solubilized monolayers to 96-well black plates and reading them in a SyQuest BioLabs Fluorescent Plate Reader (480 nm/520 nm). Data is expressed as Relative Fluorescence Units.

FIG. 3 is a bar graph representing results of the experiment designed to test the effect of TP508 on binding of metastatic colon cancer cells (KML4A) to untreated and TNF alpha-activated endothelial cell surfaces. As shown in FIG. 3, TP508 pretreatment had only a slight effect on binding of KML4A cells to control cultures (<10% decrease relative to control). In contrast, TNF alpha (TNF) increased binding of KML4A cells by approximately 8-fold. Pretreatment of the endothelial monolayers with TP508 partially inhibited the TNF alpha-induced increase in tumor cell binding (19% decrease relative to TNF alpha alone).

### Example 4

### Effect of TP508, TNF Alpha, and/or Thrombin on the Migration of KML4a Tumor Cells Through the Human Microvascular Endothelial Cells

Human microvascular endothelial cells (Cambrex BioScience; Walkersville, Md.) were plated on 48-well tissue culture plates (BD Falcon) 30,000 cells per well, and cultured in endothelial cell basal medium (EBM) with 2% fetal bovine serum (FBS) and growth factors (SingleQuots™, Clonetics, San Diego, Calif.) in 5% CO₂ at 37° C. for three days. The endothelial cell monolayers were then pretreated with saline (control) or TP508 (50 μg/ml) for eight (8) hours and then treated with saline (Control), thrombin (2 U/ml), or TNF alpha (5 ng/ml) for 16 hours followed by addition of colon metastatic KML4A tumor cells (20,000 cells per well in serum-free medium with 1% BSA) to determine the effects of co-culture. Photomicrographs were captured at various times after addition of KML4A cells using an inverted Leitz Diavert microscope (original magnification, 200×) equipped with Spot Idea™ digital camera (Diagnostic Instruments, Inc.). Micrographs were taken at the center of each well to avoid any differences in original cell density.

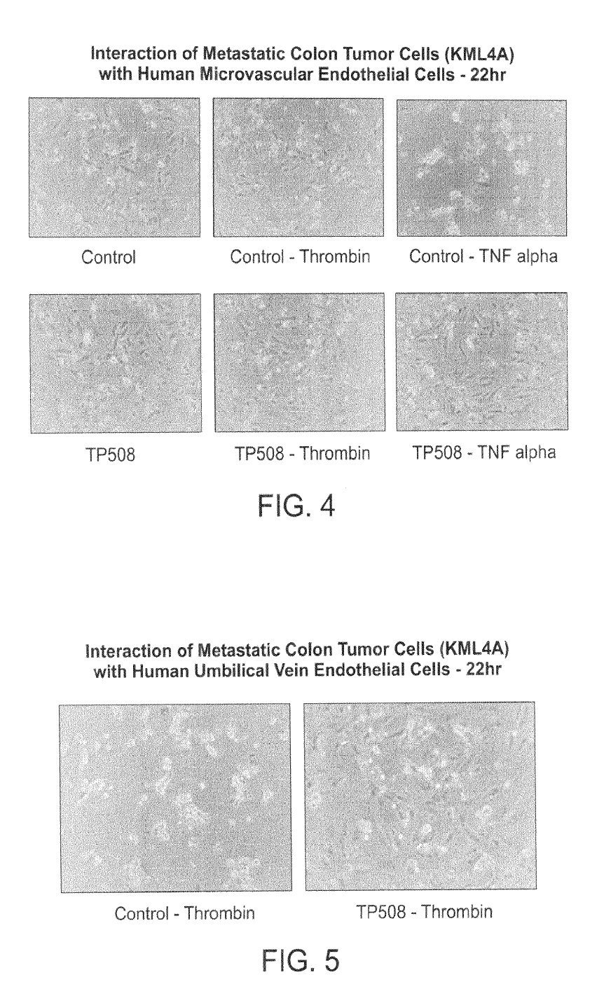

FIG. 4 is a series of pictures showing phase contrast micrographs of human microvascular endothelial cells following co-incubation with KLM4A colon cancer cells. As shown in FIG. 4, after 22 hours, control cells and those activated with thrombin or TNF alpha (top pictures) rounded tumor cells predominate while the darker endothelial cells (more cuboidal in shape) have been damaged and released from the culture substrate generating gaps between the cells. This is especially evident in cultures of cells pretreated with thrombin or TNF alpha. In contrast, cells pretreated with TP508 (bottom row) show less damage and loss of cells. These experiments demonstrate that TP508 protects the endothelial cells from tumor cell-induced changes that result in cell damage or release from the substrate.

### Example 5

### Effect of TP508 and/or Thrombin on the Migration of KML4a Tumor Cells Through the Human Umbilical Vein Endothelial Cells

Human umbilical vein endothelial cells (Cambrex BioScience; Walkersville, Md.) were plated on 48-well tissue culture plates (BD Falcon) 30,000 cells per well, and cultured in endothelial cell basal medium (EBM) with 2% fetal bovine serum (FBS) and growth factors (SingleQuots™, Clonetics, San Diego, Calif.) in 5% CO₂ at 37° C. for three days, as described in Example 4 above for microvascular endothelial cells. Endothelial cells were pre-treated with saline (control) or TP508 (50 μg/ml) for eight (8) hours and were then treated with saline or thrombin (2 U/ml) for 16 hours followed by addition of colon metastatic KML4A tumor cells (20,000 cells per well in serum-free medium with 1% BSA). Photomicrographs were captured at various times after addition of KML4A cells using an inverted Leitz Diavert microscope (original magnification, 200×) equipped with Spot Idea™ digital camera (Diagnostic Instruments, Inc.). Micrographs were taken at the center of each well to avoid any differences in original cell density.

FIG. 5 is a picture showing phase contrast micrographs of human umbilical vein endothelial cells following co-incubation with KLM4A colon cancer cells. As shown in FIG. 5, after 22 hours, in control thrombin-treated cultures there are few if any remaining endothelial cells, while in the cultures pretreated with TP508 a large portion of the endothelial cells remain attached to the culture substrate. Thus, TP508 protects the endothelial cells from tumor cell-induced damage.

### Example 6

### Effect of TP508 and/or TNF Alpha on Expression of MACC1 in Human Coronary Artery Endothelial Cells

Human coronary artery endothelial cells (Cambrex BioScience; Walkersville, Md.) were split into T-75 flasks (BD Falcon) and cultured in endothelial cell basal medium (EBM) with 2% fetal bovine serum (FBS) and growth factors (SingleQuots™, Clonetics, San Diego, Calif.) in 5% CO₂ at 37° C. Two-day post-confluent cultures were then treated with control saline (C), TNF alpha at 5 ng/ml (T), TP508 at 50 μg/ml (TP) or a combination of TNF alpha and TP508 (T+TP). After 6 hours of incubation, total RNA was isolated using RNAqueous kits (Ambion, Austin, Tex.) as described by the manufacturer. mRNA samples were reverse-transcribed using Taqman Reverse Transcription Reagents Kits (Applied Biosystems, Inc.) and analyzed by gene array analysis using H133 plus 2 chip sets (Affimetrix). All procedures and analysis of gene array data were performed in the UTMB Molecular Genomics and MicroArray Core Facility.

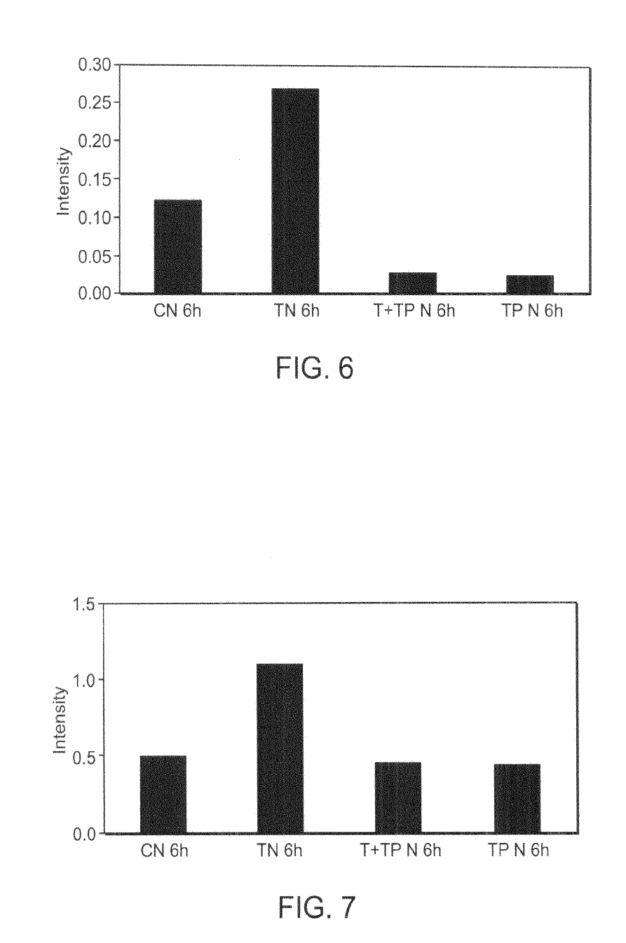

FIG. 6 shows effect of TNF alpha (T), TP508 (TP), saline (C), or combination of TNF alpha and TP508 (T+TP) on expression of mRNA for the protein known as “metastasis associated in colon cancer 1 (MACC1).” As shown in FIG. 6, TNF alpha increases endothelial cell expression of MACC1, but this increase is prevented by TP508 (reduced by ˜10-fold). Expression of MACC1 in colon cancer specimens is an independent prognostic indicator for metastasis formation and metastasis-free survival and increased expression of this protein promotes growth and metastasis of tumor cells (Stein et al. Nature Medicine 15:1, 2009). Thus, decreasing MACCCI expression should reduce metastasis.

### Example 7

### Effect of TP508 and/or TNF Alpha on Expression of MCAM in Human Coronary Artery Endothelial Cells

Human coronary artery endothelial cells (Cambrex BioScience; Walkersville, Md.) were split into T-75 flasks (BD Falcon) and cultured in endothelial cell basal medium (EBM) with 2% fetal bovine serum (FBS) and growth factors (SingleQuots™, Clonetics, San Diego, Calif.) in 5% CO₂ at 37° C. Two-day post-confluent cultures were then treated with control saline (C), TNF alpha at 5 ng/ml (T), TP508 at 50 μg/ml (TP) or a combination of TNF alpha and TP508 (T+TP). After 6 hours of incubation, total RNA was isolated using RNAqueous kits (Ambion, Austin, Tex.) as described by the manufacturer. mRNA samples were reverse-transcribed using Taqman Reverse Transcription Reagents Kits (Applied Biosystems, Inc.) and analyzed by gene array analysis using H133 plus 2 chip sets (Affimetrix). All procedures and analysis of gene array data were performed in the UTMB Molecular Genomics and MicroArray Core Facility.

FIG. 7 shows effect of TNF alpha (T), TP508 (TP), saline (C), or combination of TNF alpha and TP508 (T+TP) on expression of melanoma cell adhesion molecule (MCAM). As shown in FIG. 7, TNF alpha increases endothelial cell expression of MCAM, but TP508 prevents TNF alpha-induced MCAM expression. MCAM has been shown to increase metastasis of human melanoma cells and is upregulated by thrombin receptor PAR1 activation to promote metastasis (Melnikova et al. The Journal of Biological Chemistry Papers in press, p. 1-22, published on Aug. 24, 2009). Thus reduction of MCAM expression should decrease metastasis in vivo.

### Example 8

### Effect of TP508 and/or TNF Alpha on Expression of SNAI3 in Human Coronary Artery Endothelial Cells

Human coronary artery endothelial cells (Cambrex BioScience; Walkersville, Md.) were split into T-75 flasks (BD Falcon) and cultured in endothelial cell basal medium (EBM) with 2% fetal bovine serum (FBS) and growth factors (SingleQuots™, Clonetics, San Diego, Calif.) in 5% CO₂ at 37° C. Two-day post-confluent cultures were then treated with control saline (C), TNF alpha at 5 ng/ml (T), TP508 at 50 μg/ml (TP) or a combination of TNF alpha and TP508 (T+TP). After 6 hours of incubation, total RNA was isolated using RNAqueous kits (Ambion, Austin, Tex.) as described by the manufacturer. mRNA samples were reverse-transcribed using Taqman Reverse Transcription Reagents Kits (Applied Biosystems, Inc.) and analyzed by gene array analysis using H133 plus 2 chip sets (Affimetrix). All procedures and analysis of gene array data were performed in the UTMB Molecular Genomics and Microarray Core Facility.

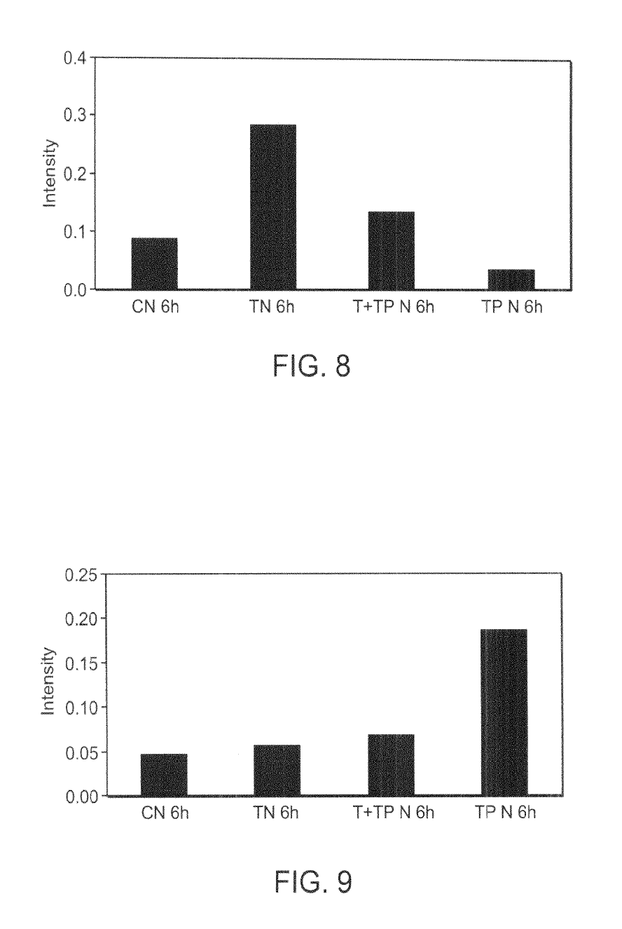

FIG. 8 shows effect of TNF alpha (T), TP508 (TP), saline (C), or combination of TNF alpha and TP508 (T+TP) on expression of snail homolog 3 (SNAI3). As shown in FIG. 8, TNF alpha increases endothelial cell expression of SNAI3, but TP508 prevents TNF alpha-induced SNAI3 expression. SNAI3 is a member of the SNAIL gene family of transcriptional regulators that play a role in the epithelial to mesenchymal transition during embryogenesis and carcinogenesis by repressing expression of E-cadherin allowing for increased motility and or invasiveness due to decreased cell-cell adhesion (Katoh et al. International Journal of Molecular Medicine 22:271-275, 2008). Thus, decreasing SNAI3 increases cell adhesion and reduces transition of cells into an invasive or metastatic phenotype.

### Example 9

### Effect of TP508 and/or TNF Alpha on Expression of BMP9 in Human Coronary Artery Endothelial Cells

Human coronary artery endothelial cells (Cambrex BioScience; Walkersville, Md.) were split into T-75 flasks (BD Falcon) and cultured in endothelial cell basal medium (EBM) with 2% fetal bovine serum (FBS) and growth factors (SingleQuots™, Clonetics, San Diego, Calif.) in 5% CO₂ at 37° C. Two-day post-confluent cultures were then treated with control saline (C), TNF alpha at 5 ng/ml (T), TP508 at 50 μg/ml (TP) or a combination of TNF alpha and TP508 (T+TP). After 6 hours of incubation, total RNA was isolated using RNAqueous kits (Ambion, Austin, Tex.) as described by the manufacturer. mRNA samples were reverse-transcribed using Taqman Reverse Transcription Reagents Kits (Applied Biosystems, Inc.) and analyzed by gene array analysis using H133 plus 2 chip sets (Affymetrix). All procedures and analysis of gene array data were performed in the UTMB Molecular Genomics and MicroArray Core Facility.

FIG. 9 shows effect of TNF alpha (TN), TP508 (TP), saline (C), or combination of TNF alpha and TP508 (T+TP) on expression of bone morphogenetic protein 9 (BMP9; also known as growth differentiation factor 2). As shown in FIG. 9, TP508 increases expression of BMP9, where as, TNF alpha does not. BMP9 is under-expressed or absent in prostate cancer. Increasing the expression of BMP9 prevents in vitro growth, cell matrix adhesion, invasion and migration of prostate cancer cells and induces prostate cancer cell apoptosis (Ye et al. Mol Cancer Res. October; 6(10):1594-606, 2008). Thus, BMP9 functions as a tumor suppressor in prostate cancer and the increased expression of this molecule may help suppress tumor cell growth and metastasis.

### Example 10

Table 1 lists examples of a wide spectrum of cancer-related proteins that have their expression affected by treatment with TP508. These genes were identified in gene microarray analysis (Affymetrics U133 plus 2) as cancer-related using GeneSifter software, and that had greater than two-fold positive or negative changes in mRNA expression induced by TP508 treatment. Table 1 includes proteins associated with or shown to be causally related to a number of different tumors. Specific types of cancer represented in this group of molecules affected by TP508 include: lung cancer, bladder cancer, esophageal cancer, prostate cancer, breast cancer, brain/testis cancers, colon, colorectal and gastric cancers, leukemia, melanoma, squamous cell carcinomas, and thyroid adenomas. In each case listed, TP508 either decreases expression of proteins that are highly expressed in tumors or involved in tumor progression or metastasis, or increases the expression of proteins thought to be involved in tumor suppression, inhibition of metastasis, and those that are deleted or under-expressed in tumor cells.

TABLE 1

| | | Effect of |
|---|---|---|
| | Effect | TP508 on |
| | on | gene |
| Gene Product Name | Cancer | expression |
| Prostate and breast cancer overexpressed | ↑ | ↓ |
| (PBOV1) | | |
| Colon Cancer Clone PM102 | ↑ | ↓ |
| Deleted in lung and esophageal cancer | ↓ | ↑ |
| Paraneoplastic associated brain -testis | ↑ | ↓ |
| cancer | | |
| Metastasis associated in colon cancer 1 | ↑ | ↓ |
| Metastasis related protein (MB3) | ↑ | ↓ |
| Metastasis suppressor 1 | ↓ | ↑ |
| E cadherin | ↓ | ↑ |
| Protocadherin 7 | ↓ | ↑ |
| Protocadherin 9 | ↓ | ↑ |
| FGF-3 (murine mammary tumor virus | ↑ | ↓ |
| integration site) | | |
| B Cell CLL/lymphoma 2 | ↑ | ↓ |
| Deleted in lympocytic leukemia 7 | ↓ | ↑ |
| Hepatic leukemia factor | ↑ | ↓ |
| MYST histone acetyltransferase | ↑ | ↓ |
| (monocytic leukemia) | | |
| TAX 1 (human T cell leukemia virus Type | ↑ | ↓ |
| 1) binding protein | | |
| Erythroblastic leukemia viral (v-erb-a) | ↑ | ↓ |
| oncogene | | |
| Breast Cancer amplified sequence 1 | ↑ | ↓ |
| CD 24 (small cell lung carcinoma cluster 4 | ↑ | ↓ |
| antigen) | | |
| Deleted in colorectal carcinoma | ↓ | ↑ |
| Human papillary thyroid carcinoma- | ↑ | ↓ |
| encoded protein | | |
| PDZK1 Interacting Protein (endothelial | ↓ | ↑ |
| integrity) | | |
| Ras associated (Ral/GDS/AF-6) domain 8 | ↓ | ↑ |
| Squamous cell carcinoma antigen | ↑ | ↓ |
| recognized by T cells | | |
| Suppression of tumorigenicity (colon | ↓ | ↑ |
| cancer) | | |
| B melanoma antigen (BAGE) | ↑ | ↓ |
| Melanoma antigen family (A, B & C | ↑ | ↓ |
| families) | | |
| Melanoma inhibitory activity family 3 | ↓ | ↑ |
| MRNA, differentially expressed in | ↑ | ↓ |
| malignant melanoma | | |
| T cell receptor alpha, anti-melanoma | ↓ | ↑ |
| Secreted frizzled-related protein 2 | ↑ | ↓ |
| Thyroid adenoma associated protein | ↑ | ↓ |
| MCF2 transforming sequence | ↑ | ↓ |
| Cancer susceptibility candidate 2 | ↑ | ↓ |
| Cancer/testis antigen family 45 number 3 | ↑ | ↓ |
| Hepatocellular carcinoma-related | ↑ | ↓ |
| Breast cancer Suppressor element CP1 | ↓ | ↑ |
| FAT tumor suppressor homolog 2 | ↓ | ↑ |

1. A method of treating cancer in a subject in need thereof, the method comprising administering to the subject a therapeutically effective amount of an agonist of a non-proteolytically activated thrombin receptor.

2. The method of claim 1, wherein the method comprises reducing or preventing metastasis of cancer in a subject in need thereof.

3. (canceled)

4. The method of claim 1, wherein the agonist is a thrombin peptide derivative comprising the amino acid sequence Asp-Ala-R, wherein R is a serine esterase conserved sequence, and wherein the thrombin peptide derivative is a 12 to 23 amino acid polypeptide.

5-11. (canceled)

12. The method of claim 4, wherein the thrombin peptide derivative comprises an N-terminus which is unsubstituted, and a C-terminus which is unsubstituted or a C-terminal amide represented by —C(O)NH₂.

13. (canceled)

14. The method of claim 4, wherein the thrombin peptide derivative comprises the polypeptide Arg-Gly-Asp-Ala-Cys-X₁-Gly-Asp-Ser-Gly-Gly-Pro-X₂-Val (SEQ ID NO: 1), wherein X₁ is Glu or Gln and X₂ is Phe, Met, Leu, His or Val.

15-17. (canceled)

18. The method of claim 4, wherein the thrombin peptide derivative comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), an N-terminal truncated fragment of the thrombin peptide derivative having at least fourteen amino acid residues, or a C-terminal truncated fragment of the thrombin peptide derivative having at least eighteen amino acid residues, wherein X₁ is Glu or Gln and X₂ is Phe, Met, Leu, His or Val.

19. The method of claim 18, wherein the thrombin peptide derivative is H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3).

20-29. (canceled)

30. The method of claim 4, wherein the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5) or a fragment thereof comprising amino acid residues 10-18 of SEQ ID NO:5, wherein Xaa is alanine, glycine, serine or an S-protected cysteine; X₁ is Glu or Gln; and X₂ is Phe, Met, Leu, His or Val.

31. (canceled)

32. The method of claim 30, wherein the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5), wherein Xaa is alanine, glycine, serine, or an S-protected cysteine, X₁ is Glu or Gln, and X₂ is Phe, Met, Leu, His or Val.

33-34. (canceled)

35. The method of claim 32, wherein the thrombin peptide derivative is the polypeptide H-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-NH₂ (SEQ ID NO:22) or H-Ala-Gly-Tyr-Lys-Pro-Asp-Glu-Gly-Lys-Arg-Gly-Asp-Ala-Ser-Glu-Gly-Asp-Ser-Gly-Gly-Pro-Phe-Val-NH₂ (SEQ ID NO:28).

36. The method of claim 1, wherein the agonist is a peptide dimer comprising two thrombin peptide derivatives 12 to 23 amino acid residues in length which, independently, comprise the polypeptide Asp-Ala-Cys-X₁-Gly-Asp-Ser-Gly-Gly-Pro-X₂-Val (SEQ ID NO: 10), wherein X₁ is Glu or Gln and X₂ is Phe, Met, Leu, His or Val, said thrombin peptide derivatives optionally comprising a C-terminal amide; and said thrombin peptide derivatives optionally comprising an acylated N-terminus.

37. The method of claim 36, wherein the dimer is essentially free of monomer, the thrombin peptide derivatives are the same, the thrombin peptide derivatives are covalently linked through a disulfide bond and the thrombin peptide derivatives consist of from about 12 to about 23 amino acids.

38-41. (canceled)

42. The method of claim 36, wherein the thrombin peptide derivatives each comprise an N-terminus which is unsubstituted; and a C-terminus which is unsubstituted or a C-terminal amide represented by —C(O)NH₂.

43-46. (canceled)

47. The method of claim 42, wherein the thrombin peptide derivatives comprise the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), wherein X₁ is Glu or Gln and X₂ is Phe, Met, Leu, His or Val; or a fragment of the polypeptide comprising amino acid residues 10-18 of SEQ ID NO:2.

48. The method of claim 47, wherein the thrombin peptide derivatives comprise the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), wherein X₁ is Glu or Gln and X₂ is Phe, Met, Leu, His or Val.

49. The method of claim 48, wherein X₁ is Glu and X₂ is Phe (SEQ ID NO: 29).

50-52. (canceled)

53. The method of claim 1, wherein the agonist is a peptide dimer represented by the following structural formula (both core peptides disclosed as SEQ ID NO: 3):

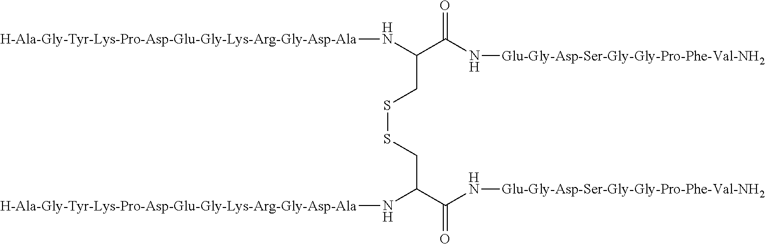

54-61. (canceled)

62. The method of claim 19, wherein the cancer is colon cancer.

63. (canceled)


SEQUENCE LISTING (29 sequences)

SEQ ID NO: 1 - protein, 14 aa, Artificial Sequence
  Description of Artificial Sequence: Synthetic peptide
  MOD_RES (6)..(6): Glu or Gln
  MOD_RES (13)..(13): Phe, Met, Leu, His or Val
RGDACXGDSGGPXV

SEQ ID NO: 2 - protein, 23 aa, Artificial Sequence
  Description of Artificial Sequence: Synthetic peptide
  MOD_RES (15)..(15): Glu or Gln
  MOD_RES (22)..(22): Phe, Met, Leu, His or Val
AGYKPDEGKRGDACXGDSGGPXV

SEQ ID NO: 3 - protein, 23 aa, Artificial Sequence
  Description of Artificial Sequence: Synthetic peptide
  N-term H; C-term NH2
AGYKPDEGKRGDACEGDSGGPFV

SEQ ID NO: 4 - protein, 14 aa, Artificial Sequence
  Description of Artificial Sequence: Synthetic peptide
  MOD_RES (5)..(5): Ala, Gly, Ser or S-protected Cys
  MOD_RES (6)..(6): Glu or Gln
  MOD_RES (13)..(13): Phe, Met, Leu, His or Val
RGDAXXGDSGGPXV

SEQ ID NO: 5 - protein, 23 aa, Artificial Sequence
  Description of Artificial Sequence: Synthetic peptide
  MOD_RES (14)..(14): Ala, Gly, Ser or S-protected Cys
  MOD_RES (15)..(15): Glu or Gln
  MOD_RES (22)..(22): Phe, Met, Leu, His or Val
AGYKPDEGKRGDAXXGDSGGPXV

SEQ ID NO: 6 - protein, 23 aa, Artificial Sequence
  Description of Artificial Sequence: Synthetic peptide
AGYKPDEGKRGDACEGDSGGPFV

SEQ ID NO: 7 - protein, 10 aa, Artificial Sequence
  Description of Artificial Sequence: Synthetic peptide
EGKRGDACEG

SEQ ID NO: 8 - protein, 33 aa, Artificial Sequence
  Description of Artificial Sequence: Synthetic polypeptide
  MOD_RES (15)..(15): Glu or Gln
  MOD_RES (22)..(22): Phe, Met, Leu, His or Val
AGYKPDEGKRGDACXGDSGGPXVMKSPFNNRWY

SEQ ID NO: 9 - protein, 33 aa, Artificial Sequence
  Description of Artificial Sequence: Synthetic polypeptide
  N-term H; C-term NH2
AGYKPDEGKRGDACEGDSGGPFVMKSPFNNRWY

SEQ ID NO: 10 - protein, 12 aa, Artificial Sequence
  Description of Artificial Sequence: Synthetic peptide
  MOD_RES (4)..(4): Glu or Gln
  MOD_RES (11)..(11): Phe, Met, Leu, His or Val
DACXGDSGGPXV

SEQ ID NO: 11-12 - skipped (2 entries)

SEQ ID NO: 13 - protein, 12 aa, Artificial Sequence
  Description of Artificial Sequence: Synthetic peptide
  MOD_RES (2)..(2): Ala or Ser
  MOD_RES (4)..(4): Glu or Gln
  MOD_RES (11)..(11): Phe, Met, Leu, His or Val
DXCXGDSGGPXV

SEQ ID NO: 14 - protein, 10 aa, Artificial Sequence
  Description of Artificial Sequence: Synthetic peptide
CEGDSGGPFV

SEQ ID NO: 15 - protein, 10 aa, Artificial Sequence
  Description of Artificial Sequence: Synthetic peptide
  MOD_RES (2)..(2): Glu or Gln
  MOD_RES (9)..(9): Phe, Met, Leu, His or Val
CXGDSGGPXV

SEQ ID NO: 16 - protein, 4 aa, Artificial Sequence
  Description of Artificial Sequence: Synthetic peptide
RGDA

SEQ ID NO: 17 - protein, 14 aa, Artificial Sequence
  Description of Artificial Sequence: Synthetic peptide
RGDACEGDSGGPFV

SEQ ID NO: 18 - protein, 33 aa, Artificial Sequence
  Description of Artificial Sequence: Synthetic polypeptide
DNMFCAGYKPDEGKRGDACEGDSGGPFVMKSPF

SEQ ID NO: 19 - protein, 33 aa, Artificial Sequence
  Description of Artificial Sequence: Synthetic polypeptide
  MOD_RES (20)..(20): Glu or Gln
  MOD_RES (27)..(27): Phe, Met, Leu, His or Val
DNMFCAGYKPDEGKRGDACXGDSGGPXVMKSPF

SEQ ID NO: 20 - protein, 23 aa, Artificial Sequence
  Description of Artificial Sequence: Synthetic peptide
  MOD_RES (14)..(14): Ala, Gly, Ser or S-protected Cys
AGYKPDEGKRGDAXEGDSGGPFV

SEQ ID NO: 21 - protein, 23 aa, Artificial Sequence
  Description of Artificial Sequence: Synthetic peptide
AGYKPDEGKRGDAAEGDSGGPFV

SEQ ID NO: 22 - protein, 23 aa, Artificial Sequence
  Description of Artificial Sequence: Synthetic peptide
  N-term H; C-term NH2
AGYKPDEGKRGDAAEGDSGGPFV

SEQ ID NO: 23 - protein, 33 aa, Artificial Sequence
  Description of Artificial Sequence: Synthetic polypeptide
  MOD_RES (5)..(5): Ala, Gly, Ser or S-protected Cys
  MOD_RES (19)..(19): Ala, Gly, Ser or S-protected Cys
DNMFXAGYKPDEGKRGDAXEGDSGGPFVMKSPF

SEQ ID NO: 24 - protein, 33 aa, Artificial Sequence
  Description of Artificial Sequence: Synthetic polypeptide
  MOD_RES (5)..(5): Ala, Gly, Ser or S-protected Cys
  MOD_RES (19)..(19): Ala, Gly, Ser or S-protected Cys
  MOD_RES (20)..(20): Glu or Gln
  MOD_RES (27)..(27): Phe, Met, Leu, His or Val
DNMFXAGYKPDEGKRGDAXXGDSGGPXVMKSPF

SEQ ID NO: 25 - protein, 33 aa, Artificial Sequence
  Description of Artificial Sequence: Synthetic polypeptide
DNMFAAGYKPDEGKRGDAAEGDSGGPFVMKSPF

SEQ ID NO: 26 - protein, 33 aa, Artificial Sequence; identical to SEQ ID NO: 25
  Description of Artificial Sequence: Synthetic polypeptide
  N-term H; C-term NH2

SEQ ID NO: 27 - protein, 33 aa, Artificial Sequence; identical to SEQ ID NO: 9
  Description of Artificial Sequence: Synthetic polypeptide

SEQ ID NO: 28 - protein, 23 aa, Artificial Sequence
  Description of Artificial Sequence: Synthetic peptide
  N-term H; C-term NH2
AGYKPDEGKRGDASEGDSGGPFV

SEQ ID NO: 29 - protein, 23 aa, Artificial Sequence
  Description of Artificial Sequence: Synthetic peptide
AGYKPDEGKRGDACEGDSGGPFV